(19) United States
(12) Patent Application Publication
(10) Pub. No.: US 2008/0280783 A1
(43) Pub. Date: Nov. 13, 2008

(54) Baculovirus-Based Gene Libraries

(76) Inventors: Kari Juhani Airenne, Kuopio (FI); Seppo Yla-Herttuala, Kuopio (FI); Olli Laitinen, Kuopio (FI)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/666,837

(22) PCT Filed: Nov. 3, 2005
(86) PCT No.: PCT/GB05/04258

§ 371 Date: Feb. 28, 2008

(30) Foreign Application Priority Data
Nov. 3, 2004 (GB) 0424356.4

(51) Int. Cl. C40B 50/00; C07H 21/00

(52) U.S. Cl. 506/23; 536/22.1

(57) ABSTRACT

A nucleotide molecule comprising a selectable gene flanked by anL1 and anL2 attachment sites of a bacteriophage and additionally comprising an origin of replication. This can be used to create a baculovirus-based gene library.

### FIELD OF THE INVENTION

This invention relates to baculovirus-based gene libraries.

### BACKGROUND OF THE INVENTION

Viral vectors are the most effective gene transfer vectors currently used in gene therapy. However, a problem is the relatively small size of foreign DNA fragments they can carry. Baculoviruses have proved to be safe and efficient gene transfer vectors, and they are able to carry considerably larger (>50 kb) insertional DNA-fragments than the traditional viral vectors.

### SUMMARY OF INVENTION

The present invention is based on the use of an attAEori element in creating baculovirus-based gene libraries.

Desired genomic fragments are converted to circular form and the attAEori element integrated into the genomic circle pool to create entry clones. The entry clone, now containing the two att-sites (attL1 and attL2), is cloned into a particular transfer plasmid, e.g. by bacteriophage lambda cloning. The transfer plasmid, containing the target DNA cassette, is further transferred into the baculovirus genome, called a bacmid, along with part of the transfer plasmid. The site-specific integration of the target DNA-cassette into bacmid can occur by Tn7-mediated transposition. Baculoviruses are produced by transfecting insect cells with this recombinant bacmid-DNA.

Existing results indicate that this method works well with plasmids sizes up to 33.5 kb, and it is to be expected that it can be used with larger plasmids. It is thus possible to transfer different libraries to baculovirus format by means of the invention.

### DESCRIPTION OF THE DRAWINGS

FIG. 1 shows the attL Adaptor Element containing attachment sites from the bacteriophage Mu.

FIG. 2 shows the p alt Aesys transposon vector.

FIG. 3 shows an overview of the different aspects of the attAe system.

### DESCRIPTION OF PREFERRED EMBODIMENTS

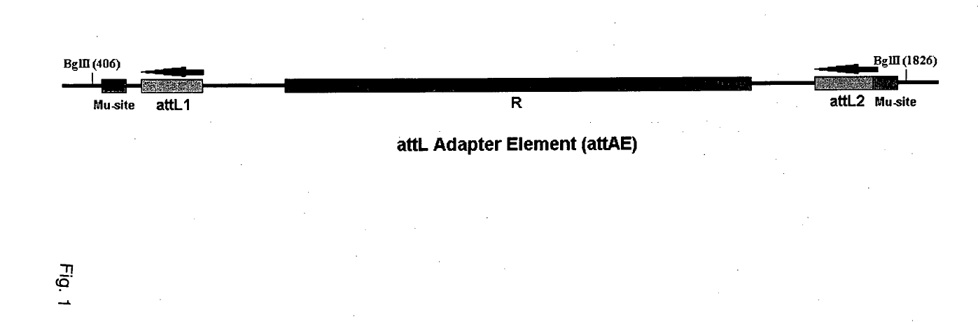

The new attAEori (attAE with an origin of replication) system provides a means to study how large DNA fragments baculoviruses can carry, e.g. up to 50, 100, 200 or 500 kb. Conversion is based on the usage of a simple and flexible attL1 & attL2 adapter element (attAE, FIG. 1), which can be incorporated into desired target DNA by various means, e.g. using conventional RE-cloning techniques (ref. 8) or taking advantage of transposase or integrase-based recombinational cloning systems (refs. 3, 4, 6, 9).

The desired genomic DNA is treated with a suitable restriction enzyme, such as NotI, an 8-cutter which generates average fragment sizes of 100 kb. The fragments may then be isolated and purified and converted to circular form.

The attAEori element may be integrated into the genomic circular DNA by a number of methods. One such method is to ligate the attAEori element directly into linearised genomic DNA (i.e. before it is converted to circular form). The preferred method is to use the MuA Transposase reaction.

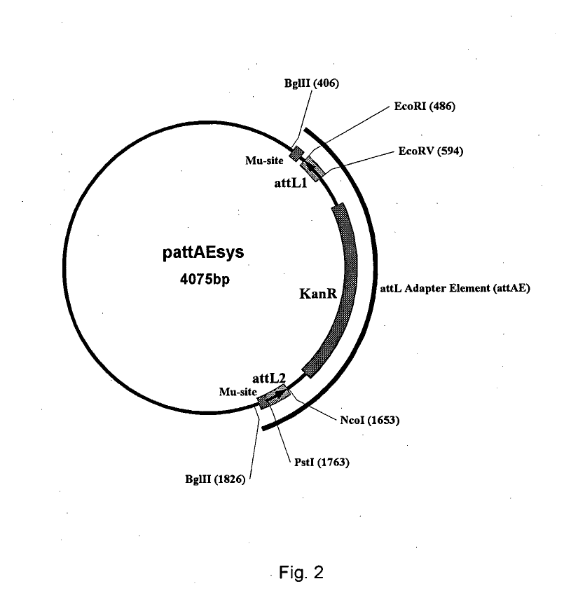

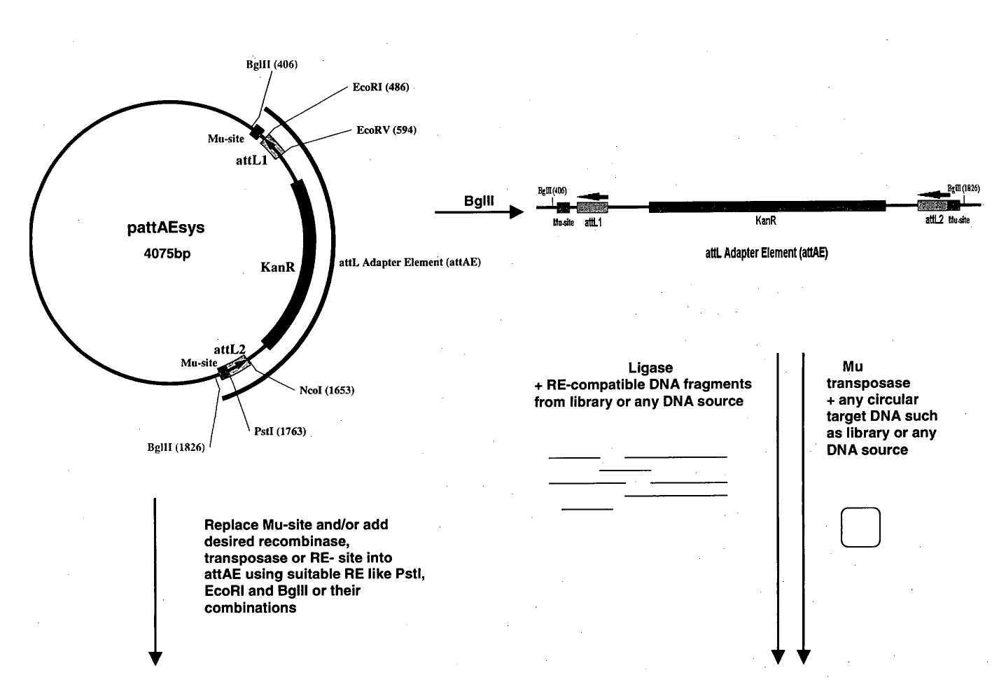

In order to take advantage of the bacteriophage Mu (ref. 3) in an in vitro random transposition reaction, catalyzed by Mu transposase protein, the attAEori is constructed into a transposon vector. The resulting vector is named for pattAEsys (FIG. 2). In the first reaction, mediated by Mu transposon, the attAEori will be integrated into target DNA in the random fashion yielding numerous different insertion clones, which can be selected using antibiotics such as kanamycin. The simple Mu reaction will thus create high diversity of bacteriophage lambda recombination system compatible entry clones (ref. 4). In a preferred embodiment, these clones can be converted effectively into baculoviruses by pBVboostFG system (ref. 1).

The two attachment sites of the bacteriophage lambda-based recombinational cloning system (attL1 and attL2), needed for the LR reaction [attL (entry clones) X attR (destination vector-like pBVboostFG)] and boosted baculovirus preparation, are cloned into the transposon vector using appropriate restriction enzymes. The attL1 element is cloned into 486 and the attL2 into 1653 site of the transposon vector, respectively, to flank the kanamycin resistance gene.

The attL1 is cloned between EcoRI and EcoRV sites of the transposon vector. For the attL2, a linker is used and cloned into the BamHI-site of the vector, into which the attL2-site is then inserted. The attL2 is cloned between the linker's NcoI and PstI-sites.

Bg/II-sites are used to free the attAE from the pattAEsys plasmid before the transposition reaction. The transposition reaction is performed with MuA-transposase, and the clones containing both target DNA and attAEori are screened using kanamycin selection. Any desired covalently closed DNA can be used as a target. Plasmids of 3-33.5 Kbp have been used successfully as model targets.

The library of entry clones may be amplified by transformation of suitable E. coli or other host cells.

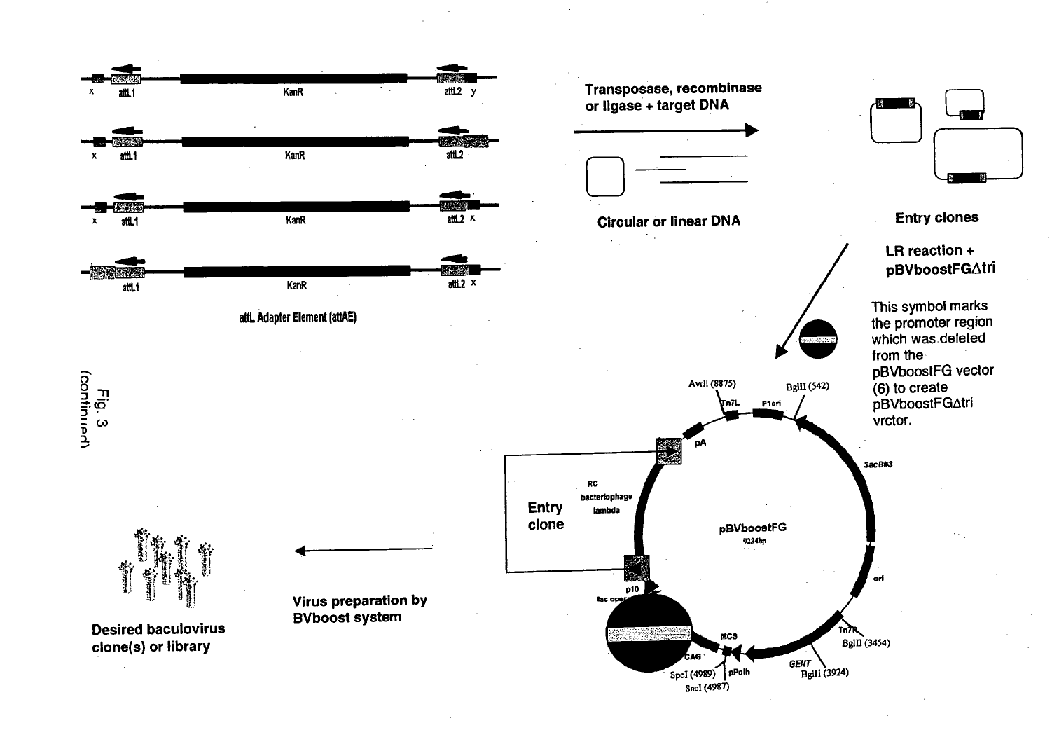

The amplified entry clones, containing now the two att-sites (attL1 and attL2) of attAE, can be converted compatible to pBVboost system (ref. 1) using pBVboostFG tri (FIG. 3). The cloning is performed using bacteriophage lambda site-specific recombination system (ref. 4). In the LR reaction the attL1 and attL2 sites, that now flank the desired target DNA, react with attR1 and attR2 sites of pBVboostFG tri. The attL1 and attL2 reacts only with attR1 and attR2, respectively. The recombination of these sites in the conversion reaction yields expression clones (donor vectors), which contains the desired target DNA in the form compatible with pBVboost system (ref. 1), allowing easy generation of baculoviruses with negligible background.

In order to produce baculoviruses, the part of the pBVboostFG tri transfer plasmid containing now the desired DNA is first converted into baculovirus genome (bacmid). This was performed by using the pBVboost Baculovirus Expression System (ref. 1). Site-specific integration of the target DNA-cassette into bacmid occurs by Tn7-mediated transposition under optimized selection protocol. Recombinant bacmids can be generated at high frequencies with a negligible background.

Baculoviruses may be produced by transfecting insect cells with recombinant bacmid-DNA. Virus production can be verified by immunoblotting using an antibody against the baculovirus major envelope glycoprotein gp64.

The results indicate that the attAEori system performs well with plasmids sizes up to 33.5 Kbp, and tests with larger plasmids are in progress. The results provide a proof of principle that the attAEori system can be used as a fast and straightforward baculovirus library construction method by incorporating attAEori into desired target DNA by transposition (MuA Transposition reaction), RE-cloning (e.g. genomic DNA) or other means (e.g. site-specific recombination). FIG. 3 gives an overview of the different aspects of the attAE system.

### REFERENCES

The following references are incorporated herein by reference.
1. Airenne et al, 2003. Nucleic Acids Res. 31:e1012.
2. Cheshenko et al, 2001. Gene Ther. 8:846-54
3. Hallet & Sherratt, 1997. FEMS Microbiol. Rev. 21:157-78
4. Hartley et al, 2000. Genome Res. 10:1788-955.
5. Kost & Condreay, 2002. Trends Biotechnol. 20:173-806
6. Lewandoski, 2001. Nat. Rev. Genet. 2:743-55
7. O'Reilly et al, 1994. Baculovirus expression vectors. A Laboratory Manual, Oxford University Press, New York
8. Sambrook et al, 1989. Molecular Cloning. A laboratory manual pp. 1-545. New York: Cold Spring Harbor Laboratory Press
9. Sauer, 1998. Methods 14:381-92

1. A nucleotide molecule comprising a selectable gene flanked by attL1 and attL2 attachment sites of a bacteriophage and additionally comprising an origin of replication.

2. The molecule according to claim 1, wherein the bacteriophage is Mu.

3. An entry clone comprising a nucleotide molecule comprising a selectable gene flanked by attL1 and attL2 attachment sites of a bacteriophage and additionally comprising an origin of replication, wherein said entry clone further comprises foreign DNA.

4. Use of an entry clone to create a baculovirus-based gene library, wherein said entry clone comprises a nucleotide molecule comprising a selectable gene flanked by attL1 and attL2 attachment sites of a bacteriophage and additionally comprising an origin of replication, wherein said entry clone further comprises foreign DNA.

5. The entry clone, according to claim 3, wherein the bacteriophage is Mu.

6. The method of use, according to claim 4, wherein the bacteriophage is Mu.
